(19) United States
(12) Patent Application Publication
(10) Pub. No.: US 2004/0048283 A1
(43) Pub. Date: Mar. 11, 2004

(54) Novel method for screening bacterial transcription modulators

(76) Inventors: Bernard Pau, Montpellier (FR); Jean-Paul Leonetti, Castelnau Le Lez (FR); Joelle Rouby, Castelnau Le Lez (FR)

Correspondence Address: YOUNG & THOMPSON, 745 SOUTH 23RD STREET 2ND FLOOR, ARLINGTON, VA, 22202

(21) Appl. No.: 10/432,987

(22) PCT Filed: Nov. 27, 2001
(86) PCT No.: PCT/FR01/03749

(30) Foreign Application Priority Data
Nov. 28, 2000 (FR) 00/15332

(51) Int. Cl. C12Q 1/68

(52) U.S. Cl. 435/6

(57) ABSTRACT

A method for detecting a compound modulating complexing between RNA polymerase and a protein intervening during transcription, which consists in: incubating a mixture comprising RNA polymerase intervening during transcription and the compound to be detected; detecting, by a complexing test, the possible significant variation of the amount of complex formed between RNA polymerase and the protein relative to a control value corresponding to the amount of complex formed between RNA polymerase and the protein in the absence of any modulator; and deducing therefrom, when there is a significant variation as defined above, that there has been formation of a bond between the compound and RNA polymerase and/or the protein intervening during transcription, which results in a modulation of the complexing between RNA polymerase and the protein intervening during transcription.

The invention relates to a new process for screening bacterial transcription modulators, in particular activators and inhibitors. The invention relates in particular to a process for screening activators and inhibitors of the binding of transcription factors with RNA polymerase. The invention also relates to a kit for the detection of bacterial transcription modulators as well as the use of this screening process in the discovery of antibiotics, antiviral and anticancer medicaments.

The transcription of genes to corresponding RNA molecules is a complex process catalyzed by RNA polymerase, dependent on the DNA, which involves a number of proteins.

Bacterial RNA polymerase is presented in two forms: the core enzyme and the holoenzyme, which appears following the fixation of the sigma (a) transcription factor onto the core enzyme. It is this holoenzyme which recognizes and binds to the promoter, allowing transcription initiation starting with a specific site (Burgess et al., 1969; Reznikoff et al., 1985). The core enzyme is incapable of recognizing the promoter sequences; it is therefore the addition of a σ factor which specifies the location of the transcription initiation. This complexation between the σ factor and the core RNA polymerase is indispensable during the first stages of bacterial transcription. After these initiation stages, σ leaves the core enzyme and other proteins, such as NusA, bind to the core enzyme.

Generally, the σ factors belong to a family of proteins which have the same functions: these are RNA polymerase subunits, necessary for transcription initiation; these factors are of primary significance with regard to the selection of the enzyme's binding sites at the level of the promoters.

The NusA factors combine with the RNA polymerase and promote transcription pauses or termination at the level of certain DNA sequences.

“Transcription pauses” is the standard definition of a slowing down or temporary stopping of enzyme activity.

The search for new targets for antibiotics is a priority in order to keep ahead of the increasingly frequent appearance of bacteria which are resistant or multiresistant to commercial antibiotics (Courvalin, 1996).

Prokaryotic RNA polymerase is an important target. In fact, it is vital to the bacterium and it is already the target of antibiotics used in therapeutics (rifampicin and derivatives). It is also a target for a number of microorganisms (Yang et al., 1995) and bacteriophages (Kolesky et al., 1999) which combat bacteria. The search for new targets on the polymerase is therefore feasible and desirable.

RNA and DNA polymerases, as well as the eukaryotic, prokaryotic and viral reverse transcriptases are good targets in order to affect the functioning of a living organism. These enzyme activities are generally relatively easy to monitor; they have therefore become targets of choice for research into new antiviral, anticancer or antibiotic drugs. This intensive industrial activity has generated faster and more reliable activity tests, which can be adapted to the new requirements of high-throughput screening.

Thus, U.S. Pat. No. 5,635,349 in the name of Tularik describes a method for the identification of a polymerase activity inhibitor, in particular RNA polymerase, derived for example from an infectious pathogenic organism. This method consists of measuring the RNA polymerase activity in the presence of various molecules the ability of which to inhibit the enzyme activity is tested. At present, the basic technique used by most laboratories consists of measuring the incorporation of radioactive nucleotides, providing evidence of enzyme activity, in the presence of the potential inhibitors (Wu et al., 1997). This method generally makes it possible to identify all the transcription inhibitors (specific inhibitors such as rifampicin, or less specific inhibitors such as intercalating agents, divalent ion chelators etc.).

However, these activity tests are expensive and they are distorted for example by the presence of RNAases and of DNAases, as well as agents interacting with DNA.

The Patent WO 96/38478 mentions a process for the detection of compounds which have the ability to inhibit the combination of a sigma sub-unit with the RNA polymerase of Mycobacterium tuberculosis; said method comprises bringing a compound into contact with the sigma sub-unit and the RNA polymerase, and the detection of the complex formed between the RNA polymerase and the sigma sub-unit. In this case, the method of detection takes place by chromatography or by immunoprecipitation according to Lesley et al. (1989) but these detection techniques are not fast enough to carry out high throughput screening.

At present, none of the high throughput screening processes in the prior art allows identification of a compound which specifically affects a transcription stage for which there is not yet any previously described inhibitor. At present, none of the processes in the prior art allows the easy identification of modulators of the bond between the sigma factor and RNA polymerase, which cannot be identified by in vitro transcription. In fact, during in vitro transcription, the complex between the sigma factor and the RNA polymerase being already formed, it is not possible to determine the modulators of the bond between the two molecules, as this bond has already been formed, before the intervention of the potential modulators.

The invention in particular makes it possible to provide a solution to these problems.

The aim of the invention is to provide a fast new, industrially applicable process, allowing the screening of products intervening during the transcription.

The aim of the invention is to provide such a process which can be used in high throughput screening.

The aim of the invention is to provide a new complexation test in which the RNAases and the DNAases do not interfere with measurement of the transcriptional activity, by degrading the DNA matrix or the RNA produced.

The aim of the invention is to provide a new process which, by targeting the complexation interface between two proteins, for example RNA polymerase and the sigma factor, makes it possible to limit the risks of resistance by mutation of the target.

The invention relates to a process for the detection of a compound modulating the complexation between RNA polymerase and a protein intervening during the transcription, in which:

a mixture comprising RNA polymerase, a protein intervening during the transcription and the compound subjected to the detection process are incubated, the incubation stage being carried out under conditions allowing:

the formation of a complex between the RNA polymerase and said protein and,

the formation of a bond, on the one hand between the said compound and on the other hand the RNA polymerase, and/or the protein intervening during the transcription,

by means of a complexation test, any significant variation in the quantity of complex formed between the RNA polymerase and said protein with respect to a control value corresponding to the quantity of complex formed between the RNA polymerase and said protein in the absence of any modulator is detected, and

when there is a significant variation as defined above, it is deduced from this, that a bond has been formed between on the one hand said compound and on the other hand the RNA polymerase, and/or the protein intervening during the transcription, which is translated by a modulation of the complexation between the RNA polymerase and the protein intervening during the transcription.

By “protein intervening during the transcription” is meant any protein factor which physically interacts with the RNA polymerase (α₂, β, β) and modifies its transcriptional activity.

By “compound modulating the complexation between RNA polymerase and a protein intervening during the transcription” is meant:

either a compound which causes a reduction in the quantity of complex formed between the RNA polymerase and the protein intervening during the transcription,

or, conversely a compound which causes an increase in the quantity of complex formed between the RNA polymerase and the protein intervening during the transcription.

In the following, by “partners” is meant the two elements which constitute the complex. By “first partner” is meant that which appears first in the mixture and by “second partner” is meant that which intervenes chronologically after the first partner. In the very particular case of the simultaneous addition of the two elements which constitute the complex, it goes without saying that the two partners correspond indiscriminately to the first partner and second partner.

By “complexation test” is understood a technique for quantitative revelation of the complex formed between the RNA polymerase and the protein intervening during the transcription.

Advantageously, this test involves the molecular marking of at least one of the partners, namely the RNA polymerase and/or said protein, by a substance. This marking allows a direct or indirect quantitative physical measurement, by signal emission or consumption, spontaneously or after the addition of a substrate or signal.

Advantageously, this test does not involve the presence, for the complex formed between the RNA polymerase and said protein, of a particular physico-chemical property, such as molecular size or isoelectric point. Consequently, this test differs from chromatography, in that it is a filtration/exclusion or charge effect technique.

The marking, as mentioned above, can be carried out using, in particular, a radioactive element, a fluorescent element, a luminescent element, an enzyme, biotin for an indirect revelation by marked avidin, etc.

In a particular embodiment, when the two partners, namely the RNA polymerase and said protein, can be marked by substances capable of energy exchanges (transfers) between themselves, the determination of the quantity of complex (or of its variation) can be carried out in solution; the formation of the complex is accompanied by the bringing together of the two partners, which allows the transfer of energy between the two markers and then leads to an increase or reduction in the intensity of the fluorescence signal emitted by one of the two markers.

In another embodiment, only one of the partners is marked and the other is immobilized on a solid phase, either before being brought into contact with the marked partner, or subsequently. The immobilization can of a physico-chemical kind, such as for example, by adsorption on a hydrophobic plastic surface, or of a bio-specific kind: in this case, a biological attractor, which can be an antibody specific to one of the partners or avidin capable of immobilizing the partner previously coated with biotin, is itself previously immobilized. In all cases, it is the second partner which makes it possible to determine the quantity of complex or the variation in the quantity of complex formed between the RNA polymerase and said protein by quantitative revelation of its marker, which can have been fixed by a permanent chemical bond (radioactive element, fluorescent element, luminescent element, enzyme, biotin etc.) or be introduced in bio-specific manner. In this case, it is possible to use an antibody to the second partner if it is directly or indirectly marked, or directly or indirectly marked avidin.

Moreover, this test is also independent of the immunological techniques known as ELISA (enzyme-linked immunosorbent assay), since it uses an antibody only in order to reveal one of the partners of a bio-specific interaction to which this antibody is alien. This test is based on the interaction between the RNA polymerase and said protein, in contrast to an ELISA test, which is based on an antigen-antibody interaction. Moreover, in this test, the antibody is only used for the detection and can be replaced, for example, by a fluorescent marker or radioactive label. This test also differs from immunoprecipitation as the antibody is not used in order to immunoprecipitate a complex formed between the RNA polymerase and the protein intervening during the transcription: it serves either to capture this complex on a solid phase, or to reveal one of the partners of the complex.

In order to obtain the control value, corresponding to an absence of modulation, the following experiment is carried out:

a mixture comprising the RNA polymerase and a protein as defined above is incubated under conditions allowing the formation of a complex between the RNA polymerase and said protein, and

the quantity of complex formed between the RNA polymerase and said protein is detected; this quantity corresponding to said control value.

By “significant variation in the quantity of complex formed between the RNA polymerase and the protein intervening during the transcription”, is meant a variation of approximately more than 20% of the quantity of complex formed, and preferably of at least approximately 50%.

The invention relates to a detection process as defined above, in which the modulating compound is a compound activating the complexation between the RNA polymerase and a protein intervening during the transcription, and in which:

a mixture comprising the RNA polymerase, a protein intervening during the transcription and the compound subjected to the detection process is incubated, the incubation stage being carried out under conditions allowing:

the formation of a complex between the RNA polymerase and said protein and,

optionally the formation of a bond on the one hand between said compound and on the other hand the RNA polymerase, and/or the protein intervening during the transcription,

by means of a complexation test, any significant variation in the quantity of complex formed between the RNA polymerase and said protein with respect to a control value corresponding to the quantity of complex formed between the RNA polymerase and said protein in the absence of any activator is detected, and

when there is a significant variation as defined above, it is deduced from this that a bond has been formed between on the one hand said compound and on the other hand the RNA polymerase and/or the protein intervening during the transcription, which is translated by an activation of the complexation between the RNA polymerase and the protein intervening during the transcription.

By “compound activating the complexation between the RNA polymerase and a protein intervening during the transcription”, is meant a compound which causes an increase in the quantity of complex.

Said activating compound causes a greater complexation, i.e. an increase of at least 120% and preferably greater than 150% with respect to the control (percentage of 100%) corresponding to the quantity of complex formed between the RNA polymerase and said protein in the absence of any activator.

The invention relates to a detection process as defined above, in which the modulating compound is a compound inhibiting the complexation between the RNA polymerase and a protein intervening during the transcription, and in which:

a mixture comprising the RNA polymerase, a protein intervening during the transcription and the compound subjected to the detection process is incubated, the incubation stage being carried out under conditions allowing:

the formation of a complex between the RNA polymerase and said protein and,

optionally the formation of a bond on the one hand between said compound and on the other hand the RNA polymerase and/or the protein intervening during the transcription,

by means of a complexation test, any significant variation in the quantity of complex formed between the RNA polymerase and said protein with respect to a first control value and/or to a second control value is detected, one of these control values corresponding to the quantity of complex formed between the RNA polymerase and said protein in the absence of any inhibitor and the other of these control values corresponding to the quantity of complex formed between the RNA polymerase and said protein in the presence of a reference inhibitor, and

when there is a significant variation as defined above, it is deduced from this, that a bond has been formed between on the one hand said compound and on the other hand the RNA polymerase, and/or the protein intervening during the transcription, which is translated by an inhibition of the complexation between the RNA polymerase and the protein intervening during the transcription.

By “compound inhibiting the complexation between the RNA polymerase and a protein intervening during the transcription”, is meant a compound which causes a reduction in the quantity of complex formed between the RNA polymerase and the protein intervening during the transcription.

In order to obtain the first control value, the experiment as described above is carried out, corresponding to an absence of inhibition.

In order to obtain the second control value, corresponding to a reference inhibition, the following experiment is carried out:

a mixture comprising the RNA polymerase, a protein as defined above and the reference inhibitor is incubated, the incubation stage being carried out under conditions allowing:

the formation of a complex between the RNA polymerase and said protein and,

the formation of a bond between the reference inhibitor and the RNA polymerase,

the quantity of complex formed between the RNA polymerase and said protein is detected; this quantity corresponding to the second control value. This quantity is lower than that measured during the first control due to the formation of the complex between the reference inhibitor and the RNA polymerase, and its negative consequence on the formation of said complex.

The reference inhibitor is for example a monoclonal antibody, in particular the monoclonal antibody 3E10 (cf. example).

An advantageous detection process is a detection process as defined above comprising the use of a single control value, corresponding to the incubation of the RNA polymerase alone with the protein intervening during the transcription in the absence of any inhibitor (which corresponds to an absence of inhibition).

In order to obtain this control value, corresponding to an absence of inhibition, the experiment as described above is carried out, which comprises the incubation of the RNA polymerase alone with the protein intervening during the transcription.

An advantageous detection process is a process as defined above, comprising the use of two control values, one corresponding to the incubation of the RNA polymerase alone with the protein intervening during the transcription in the absence of any inhibitor (which corresponds to an absence of inhibition) and the other corresponding to an incubation of the RNA polymerase with the protein intervening during the transcription and with a reference inhibitor (which corresponds to a reference inhibition).

In order to obtain the two control values, the two experiments as described above are carried out; the first is obtained by detecting the quantity of complex formed between the RNA polymerase and the protein intervening during the transcription in the absence of inhibitor and the second by detecting the quantity of complex formed between the RNA polymerase and the protein intervening during the transcription in the presence of the reference inhibitor.

An advantageous detection process is a process as defined above, in solid phase, comprising the use of a first control value and/or of a second control value and/or of a third control value,

one corresponding to the quantity of complex formed between the RNA polymerase and said protein in the absence of any inhibitor, the other corresponding to the quantity of complex formed between the RNA polymerase and said protein in the presence of a reference inhibitor, and the other corresponding:

either to the absence of complex formed between the RNA polymerase and the protein intervening during the transcription, resulting from the introduction of the protein intervening during the transcription fixed on a support, of the RNA polymerase and of an excess of protein intervening during the transcription, the excess of protein intervening during the transcription preferably being previously incubated with the RNA polymerase, the absence of said complex corresponding to a total inhibition of the complexation between the RNA polymerase and said protein,

or to the absence of complex formed between the RNA polymerase and the protein intervening during the transcription, resulting from the presence of RNA polymerase alone, and from the absence of protein intervening during the transcription.

By “solid phase process”, is meant a process where one of the partners, namely the RNA polymerase or the protein intervening during the transcription, is immobilized covalently (chemical reaction) or non-covalently (non-specific adsorption on plastic, avidin-biotin system, antibody) on a solid support. The second partner is incubated under conditions allowing the complexation between the two partners, then the excess of the second partner is optionally eliminated by washing. The complex is then subjected to the detection process.

In order to obtain the first control value, the experiment is carried out as described above, corresponding to an absence of inhibition.

In order to obtain the second control value, corresponding to a reference inhibition, the quantity of complex formed between the RNA polymerase and the protein intervening during the transcription is detected, in the presence of the reference inhibitor.

In order to obtain the third control value, corresponding to a reference inhibition,

either the following experiment is carried out, corresponding to incubation of a support on which is fixed the protein intervening during the transcription with the RNA polymerase and an excess of the protein intervening during the transcription, the excess of said protein preferably being pre-incubated with the RNA polymerase, and which causes a total inhibition of the complexation between the RNA polymerase and said protein, i.e. an absence of complex formed between the RNA polymerase and said protein,

or the following experiment is carried out corresponding to incubation of a support with the RNA polymerase alone, which leads to a total absence of the complexation between the RNA polymerase and said protein, i.e. an absence of complex formed between the RNA polymerase and said protein.

An advantageous detection process is a process as defined above in liquid phase, comprising the use of a first control value and/or of a second control value and/or of a third control value,

one corresponding to the quantity of complex formed between the RNA polymerase and said protein in the absence of any inhibitor, the other corresponding to the quantity of complex formed between the RNA polymerase and said protein in the presence of a reference inhibitor, and the other corresponding to the absence of complex formed between the RNA polymerase and the protein intervening during the transcription, resulting from the introduction of the RNA polymerase, an excess of non-marked protein intervening during the transcription and of said marked protein, the absence of said complex corresponding to a total inhibition of the complexation between the RNA polymerase and said protein.

By liquid-phase process, is meant a process where the partners are in solution in a buffer solution. One or both of the partners are, for example, marked with a fluorescent molecule. Their interaction is quantified by transfer or polarization of fluorescence.

In order to obtain the first control value, the experiment is carried out as described above, corresponding to an absence of inhibition.

In order to obtain the second control value, corresponding to a reference inhibition, the quantity of complex formed between the RNA polymerase and the protein intervening during the transcription is detected, in the presence of the reference inhibitor.

In order to obtain the third control value, corresponding to a reference inhibition, the following experiment is carried out, corresponding to an incubation of the RNA polymerase, an excess of non-marked protein intervening during the transcription and the protein intervening during the transcription, which causes a total inhibition of the complexation between the RNA polymerase and said protein, i.e. an absence of complex formed between the RNA polymerase and said protein.

An advantageous detection process is a process as defined above, in which the mixture comprising the RNA polymerase, a protein intervening during the transcription and a compound subjected to the detection process is prepared:

either by simultaneously adding the RNA polymerase, the protein intervening during the transcription and the compound subjected to the detection process,

or by successively adding: the RNA polymerase, the compound subjected to the detection process and the protein intervening during the transcription, or successively: the protein intervening during the transcription, the compound subjected to the detection process and the RNA polymerase,

or by adding said compound previously incubated with the RNA polymerase or said protein, and either said protein or the RNA polymerase respectively,

or by adding said compound and the RNA polymerase previously incubated with said protein.

The preparation of the mixture comprising the RNA polymerase, a protein intervening during the transcription and a compound subjected to the detection process by simultaneously adding the RNA polymerase, the protein intervening during the transcription and the compound subjected to the detection process, can make it possible to seek compounds which bind to a complex between the RNA polymerase and said protein and which dissociate said complex as well as the compounds binding only one of the two partners, but which are sufficiently efficient to compete with a pre-formed complex.

The preparation of the mixture comprising the RNA polymerase, a protein intervening during the transcription and a compound subjected to the detection process by successively adding: the RNA polymerase, the compound subjected to the detection process and the protein intervening during the transcription, can facilitate the detection of compounds binding the RNA polymerase. This embodiment can make it possible to select, besides the RNA polymerase ligands, the best ligands of said protein which are, in this case, disadvantaged from the kinetic point of view.

The preparation of the mixture comprising the RNA polymerase, a protein intervening during the transcription and a compound subjected to the detection process by successively adding: the protein intervening during the transcription, the compound subjected to the detection process and the RNA polymerase, can promote the detection of compounds which bind the molecule intervening during the transcription, as well as the search for very good RNA polymerase ligands which are disadvantaged from the kinetic point of view.

The preparation of the mixture comprising the RNA polymerase, a protein intervening during the transcription and a compound subjected to the detection process by adding said compound previously incubated with the RNA polymerase to said protein, can facilitate the binding of said compound with the RNA polymerase before the addition of the protein intervening during the transcription. This embodiment comprising a preincubation can promote the detection of molecules which bind the RNA polymerase.

The preparation of the mixture comprising the RNA polymerase, a protein intervening during the transcription and a compound subjected to the detection process by adding said compound previously incubated with said protein to the RNA polymerase, makes it possible to facilitate the bond between said compound and the protein intervening during the transcription before the addition of the RNA polymerase. This embodiment can promote the detection of molecules which bind said protein.

The preparation of the mixture comprising the RNA polymerase, a protein intervening during the transcription and a compound subjected to the detection process by adding said compound and the RNA polymerase previously incubated with said protein, makes it possible to detect the compounds which promote the dissociation of the complex formed between the RNA polymerase and said protein.

The invention also relates to a detection process as defined above, in which, before, during or after the incubation stage, either the RNA polymerase or the protein intervening during the transcription is applied to a solid support.

This application can be carried out by intervention of a covalent (physico-chemical) or biospecific link between the solid support and the RNA polymerase or said protein.

When the RNA polymerase is applied to a solid support before the incubation stage, it is possible for the RNA polymerase to be denatured.

When the protein intervening during the transcription is applied to a solid support before the incubation stage, it is possible for said protein to be denatured.

An advantageous detection process of the invention is a process as defined above, in which the RNA polymerase and the compound subjected to the detection process are added simultaneously, not previously mixed, to the protein intervening during the transcription applied to a support.

This embodiment can make it possible to detect both the ligands of the protein intervening during the transcription, those of the RNA polymerase and also those of the complex formed between said protein and the RNA polymerase. This embodiment is very stringent for the molecules which inhibit the association between the RNA polymerase and said protein and can also make it possible to seek compounds which dissociate the complex formed between the RNA polymerase and said protein.

An advantageous detection process of the invention is a process as defined above, in which the compound subjected to the detection process previously incubated with the RNA polymerase is added to the protein intervening during the transcription, applied to a support.

This embodiment can make it possible to detect both the ligands of the protein intervening during the transcription, those of the RNA polymerase and also those of the complex formed between said protein and the RNA polymerase. This embodiment can facilitate the binding of said compound with the RNA polymerase but can also serve to select the best ligands of said protein which are, in this case, kinetically disadvantaged.

An advantageous detection process of the invention is a process as defined above, in which the protein intervening during the transcription and the compound subjected to the detection process are added simultaneously, not previously mixed, to the RNA polymerase applied to a support.

This embodiment can make it possible to detect both the ligands of the protein intervening during the transcription, those of the RNA polymerase and also those of the complex formed between said protein and the RNA polymerase. This embodiment is very stringent for the molecules which inhibit the association between the RNA polymerase and said protein and can also make it possible to seek compounds which dissociate the complex formed between the RNA polymerase and said protein.

An advantageous detection process of the invention is a process as defined above, in which the compound subjected to the detection process previously incubated with the protein intervening during the transcription is added to the RNA polymerase applied to a support.

This embodiment can make it possible to detect both the ligands of the protein intervening during the transcription, those of the RNA polymerase and also those of the complex formed between said protein and the RNA polymerase. This embodiment can thus facilitate the bond of said compound with said protein before the incubation with the RNA polymerase and makes it possible to seek ligands of said protein. It can serve to select the best ligands of the RNA polymerase which are, in this case, kinetically disadvantaged.

An advantageous detection process is a process as defined above, in which the compound subjected to the detection process and the RNA polymerase are added one after the other to the protein intervening during the transcription applied to a support.

The application of the protein intervening during the transcription to a support then the successive addition of the compound subjected to the detection process and the RNA polymerase, can make it possible to detect the compounds inhibiting only the protein intervening during the transcription. In fact, if the compound as defined above is not fixed on the protein as defined above, it is eliminated during the washing which has taken place before the addition of the RNA polymerase.

An advantageous detection process is a process as defined above, in which the compound subjected to the detection process and the protein intervening during the transcription are added one after the other to the RNA polymerase applied to a support.

The application of the RNA polymerase to a support, then the successive addition of the compound subjected to the detection process and of the protein intervening during the transcription, can make it possible to detect the compounds inhibiting only the RNA polymerase. In fact, if the compound as defined above is not fixed on the RNA polymerase, it is eliminated during the washing which takes place before the addition of the protein as defined above.

The invention relates to a detection process as defined above, in which, during the incubation stage of the RNA polymerase with a protein intervening at the time of the transcription and with a compound subjected to the detection process, a bond is formed:

either between said compound and between the RNA polymerase,

or between said compound and between said protein,

or between said compound, between said protein and between the RNA polymerase.

When a bond is formed between said compound and between the RNA polymerase, if the compound subjected to the detection process is fixed in the region of the complexation site of said protein, said compound prevents the protein intervening during the transcription from becoming complexed with the RNA polymerase whilst the fixation of said compound beside the complexation site of said protein leads to a conformation change and also prevents the complexation between said protein and the RNA polymerase.

When a bond is formed between said compound and between said protein, if said compound is fixed in the region of the complexation site of the RNA polymerase, said compound prevents the RNA polymerase from becoming complexed with said protein whilst the fixation of said compound beside the complexation site of the RNA polymerase leads to a conformation change and also prevents the complexation between said protein and the RNA polymerase.

When a bond is formed between said compound, between said protein and between the RNA polymerase, either said compound binds the RNA polymerase involved in the complex with said protein and promotes dissociation, or the compound binds said protein, changes its conformation and forces dissociation.

The invention relates to a detection process as defined above, in which, during the stage of detection of any significant variation in the quantity of complex formed between the RNA polymerase and the protein intervening during the transcription, an anti-RNA polymerase antibody is used.

The invention relates to a detection process as defined above, in which the protein intervening during the transcription has a molecular weight greater than approximately 15 kDa or is a fusion protein between a protein with a molecular weight of less than 15 kDa and another protein, such as GST (glutathione S transferase).

Examples of such proteins include in particular sigma factor domains in GST-fusion form, and the proteins Gp33 or 55 of the bacteriophage T4 in GST-fusion form.

The invention relates to a detection process as defined above, in which the RNA polymerase concentrations are comprised between approximately 1 fmole and approximately 100 pmole/test, in particular between approximately 1 fmole and approximately 10 pmole/test, those of the protein intervening during the transcription between approximately 10 fmole and approximately 500 pmole/test and those of the compound subjected to the detection process between approximately 1 μM and approximately 1 μM, in particular approximately 1 nM and approximately 1 μM.

The concentration ranges below the weakest concentration correspond to the detection limit, whilst the concentration ranges above the strongest concentration promote a non-specific bond and require too great a quantity of protein, which is highly disadvantageous with respect to the concentrations of compound subjected to the detection process, which it is necessary to add in order to observe the inhibiting effect.

The invention relates to a detection process as defined above, in which the RNA polymerase used originates from prokaryotic cells, in particular from E. coli.

The invention relates to a detection process as defined above, in which the protein intervening during the transcription intervenes either during the transcription initiation stage, or during the elongation stage, or during the transcription termination stage.

By “intervening during the transcription initiation stage”, is understood a protein which binds to the RNA polymerase, conferring upon it a promoter specificity and allowing the transcription initiation.

Examples of proteins intervening during the initiation are in particular the family of the sigma factors, in particular the factor sigma 70, as well as the proteins Gp33, Gp45 and Gp55 of the bacteriophage T4.

By “intervening during the transcription elongation stage”, is understood a protein which binds to the RNA polymerase and changes its rate of elongation.

Examples of proteins intervening during elongation are in particular the proteins NusA, greA and greB.

By “intervening during the transcription termination stage”, is understood a protein which changes the transcription termination site.

Examples of proteins intervening during the termination are in particular the proteins NusA, NusB, NusG, Rho or the protein N of bacteriophage lambda.

The invention relates to a detection process as defined above, in which the protein intervening during the transcription is:

either the protein σ⁷⁰ or an equivalent protein,

By equivalent protein, is understood any protein which binds the RNA polymerase, confers upon it a promoter specificity and allows initiation.

either the protein NusA or an equivalent protein,

By equivalent protein, is understood any protein having between 22 and 100% sequence identity with that of the protein NusA of E. coli (Swiss Prot P03003).

either fragments of one of these two proteins,

or one of these two proteins in the form of a fusion protein,

or fragments of one of these two proteins in the form of a fusion protein.

The invention relates to a detection process as defined above, in which:

the protein intervening during the transcription is adsorbed on a support,

said support is incubated with the RNA polymerase and with the compound subjected to the detection process, which leads to the formation of a complex between the RNA polymerase and said protein and the optional formation of a bond between the RNA polymerase and said compound,

said support is incubated with an anti-RNA polymerase antibody,

by means of a complexation test, any significant variation in the quantity of complex formed between the RNA polymerase and said protein, with respect to a control value corresponding to the quantity of complex formed between the RNA polymerase and said protein in the absence of any modulator is detected, and

when there is a significant variation as defined above, it is deduced from this, that a bond has been formed between said compound and the RNA polymerase, which corresponds to a modulation of the complexation between the RNA polymerase and the protein intervening during the transcription.

By “modulation of the complexation between the RNA polymerase and the protein intervening during the transcription”, is understood both the activation and the inhibition of said complexation.

The invention also relates to a detection process as defined above, in which:

the protein intervening during the transcription is adsorbed on a support,

said support is incubated with the RNA polymerase and with the compound subjected to the detection process, which leads to the formation of a complex between the RNA polymerase and said protein and the optional formation of a bond between the RNA polymerase and said compound,

said support is incubated with an anti-RNA polymerase antibody,

by means of a complexation test, any significant variation in the quantity of complex formed between the RNA polymerase and said protein, with respect to a control value corresponding to the quantity of complex formed between the RNA polymerase and said protein in the absence of any inhibitor is detected, and

when there is a significant variation as defined above, it is deduced from this, that a bond has been formed between said compound and the RNA polymerase, which corresponds to a inhibition of the complexation between the RNA polymerase and the protein intervening during the transcription.

The invention relates to a kit for the detection of a compound modulating, in particular a compound inhibiting, the complexation between the RNA polymerase and a protein intervening during the transcription comprising:

one or more proteins intervening during the transcription; said protein can be in the form of a fusion protein and is in particular:

either the protein σ⁷⁰ or an equivalent protein,

or the protein NusA or an equivalent protein,

or fragments of one of these two proteins,

the RNA polymerase,

media or buffers necessary for dilution,

optionally washing means,

media or buffers allowing the formation of a complex between the RNA polymerase and the protein intervening during the transcription and the formation of a bond between the RNA polymerase and the modulating compound,

means for the detection of the variation in the quantity of complex formed between the RNA polymerase and between the protein intervening during the transcription.

The invention also relates to a kit for the detection of a compound inhibiting the complexation between the RNA polymerase and a protein intervening during the transcription comprising:

one of more proteins intervening during the transcription,

the RNA polymerase,

media or buffers necessary for dilution,

washing means,

media or buffers allowing the formation of a complex between the RNA polymerase and the protein intervening during the transcription and the formation of a bond between the RNA polymerase and the inhibiting compound,

means for detecting the variation in the quantity of complex formed between the RNA polymerase and between the protein intervening during the transcription.

The media or buffers necessary for dilution are for example:

PBS (137 mM NaCl, 2.7 mM KCl, 4.3 mM Na₂HPO₄, 1.4 mM K₂HPO₄),

PBS Tween BSA (0.1% Tween 20, 1% BSA in PBS),

An appropriate washing means is for example PBS Tween (0.1% Tween in PBS). Thus, three washings are carried out with PBS Tween.

The means for detecting the variation in the quantity of complex formed between the RNA polymerase and between the protein intervening during the transcription are carried out for example by using:

a fluorescent antipolymerase antibody, or

an antipolymerase antibody marked with alkaline phosphatase or peroxidase, or

biotinylated polymerase, or

a radioactively-labelled antibody or radioactively-labelled proteins.

The invention relates to a kit for detection as defined above, which comprises a support on which the protein intervening during the transcription is adsorbed; said protein can be in the form of a fusion protein and is in particular:

either the protein σ⁷⁰ or an equivalent protein,

or the protein NusA or an equivalent protein,

or fragments of one of these two proteins,

The invention relates to a detection kit as defined above, which comprises a support on which the RNA polymerase is adsorbed.

FIGURES

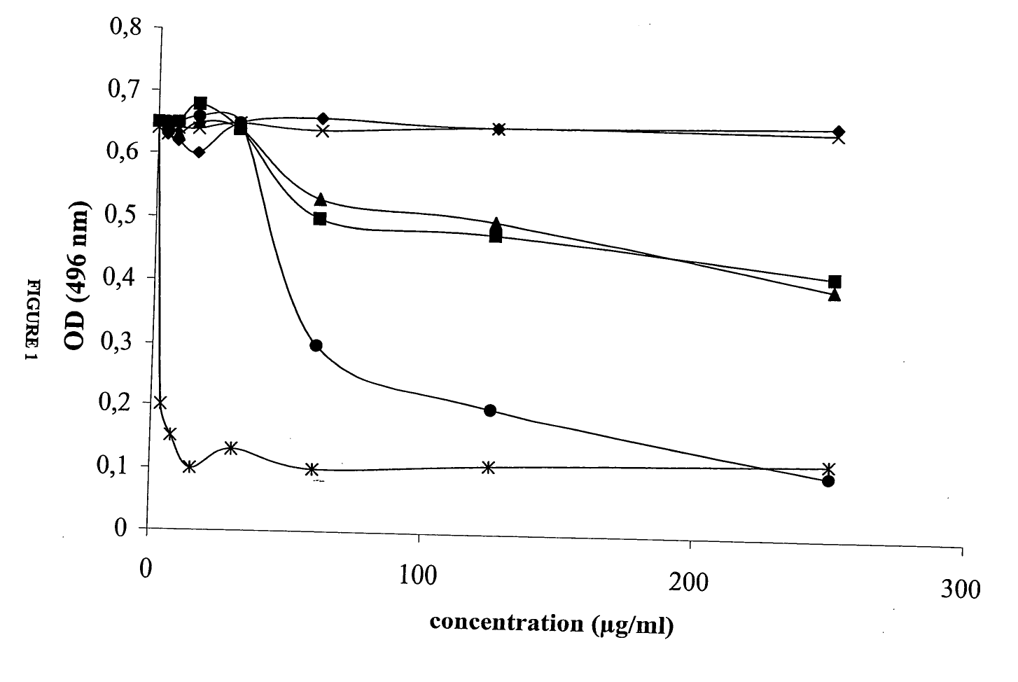

FIG. 1 corresponds to the inhibition of the bond between the core enzyme of the RNA polymerase and the protein σ⁷⁰ by the compounds of the family of 5,988,031, the chemical formulae of said compounds being represented below. Thus, the different compounds tested are incubated in the presence of the core enzyme in a microassay dish on which the protein σ⁷⁰ is adsorbed, each well of the plate containing 1 μM of σ⁷⁰ during the adsorption phase. The dish is then washed and incubated with the monoclonal antibody 11D11 marked with peroxidase, then revealed.

The y-axis represents the optical density measured at 496 nm, resulting from the inhibition of the mixture comprising the RNA polymerase core enzyme, the protein σ⁷⁰ and a compound tested, and the x-axis represents the concentration of said compounds tested in μg/ml.

The curve with the stars (*) corresponds to the optical density at 496 nm with the compound 5,988,031; the curve with the circles (•) corresponds to the optical density at 496 nm with the compound 5,951,261; the curve with the triangles (▴) corresponds to the optical density at 496 nm with the compound 5,128,773; the curve with the squares (▪) corresponds to the optical density at 496 nm with the compound 5,128,772; the curve with the diamonds (♦) corresponds to the optical density at 496 nm with the compound 5,128,767 and the curve with the crosses (x) corresponds to the optical density at 496 nm with the compound 5,210,476.

It is recalled that the bond percentage is calculated as follows:

$$\text{bond \%} = \frac{\begin{matrix}\text{absorbance in the presence of inhibitor tested} - \\ \text{minimum absorbance with an excess of reference inhibitor}\end{matrix}}{\begin{matrix}\text{maximum absorbance without inhibitor tested} - \\ \text{minimum absorbance with an excess of reference inhibitor}\end{matrix}} \times 100$$

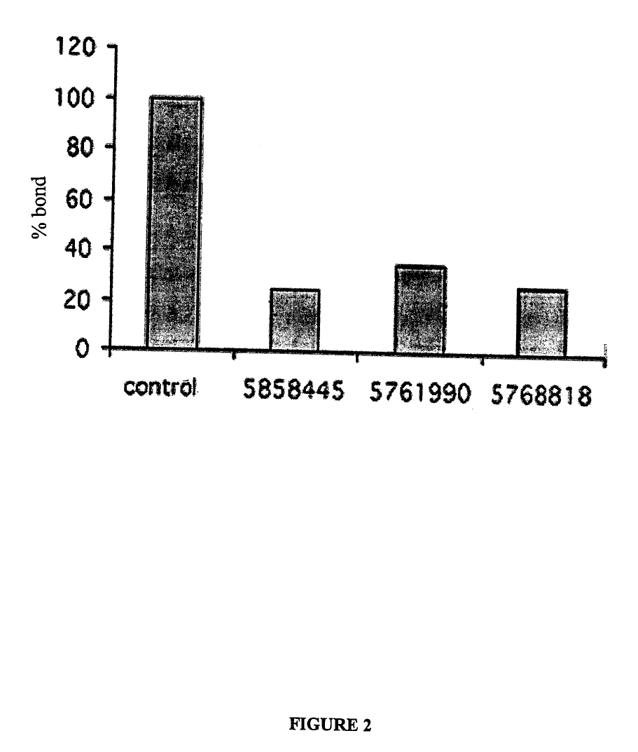

FIG. 2 represents the bond percentage between the RNA polymerase and the protein σ⁷⁰, with respect to a control value corresponding to the maximum bond (bond percentage of 100%), for the compounds 5,858,445, 5,761,990 and 5,768,818 (see formulae below).

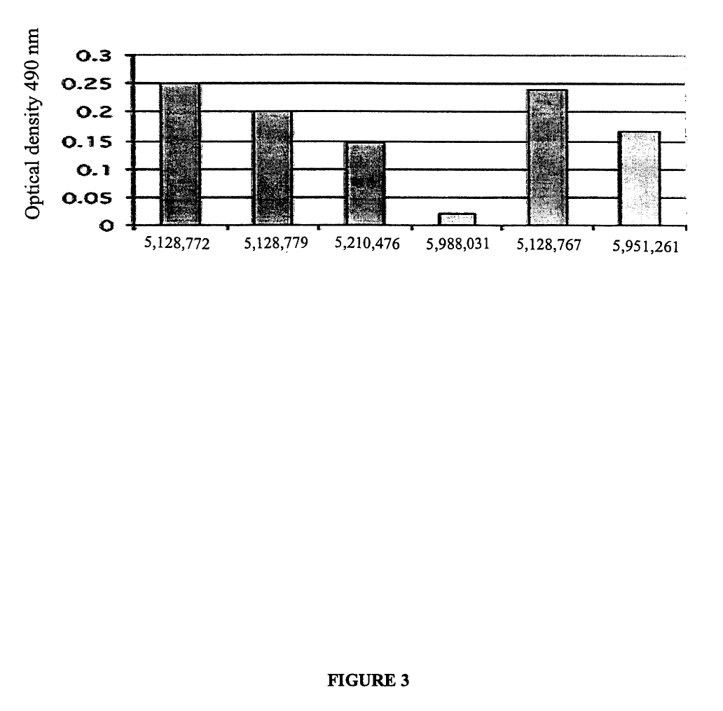

FIG. 3 represents the optical density measured at 490 nm during the introduction of RNA polymerase and of the protein NusA fixed on a support, with one of the compounds tested for its ability to inhibit the bond between the RNA polymerase and NusA.

More precisely, the different compounds tested (from the family of 5,988,031) are incubated in the presence of core enzyme in a microassay dish on which the protein NusA is adsorbed at a concentration equal to 20 μg/ml. The dish is then washed and incubated with the monoclonal antibody 11D11 marked with peroxidase, then revealed.

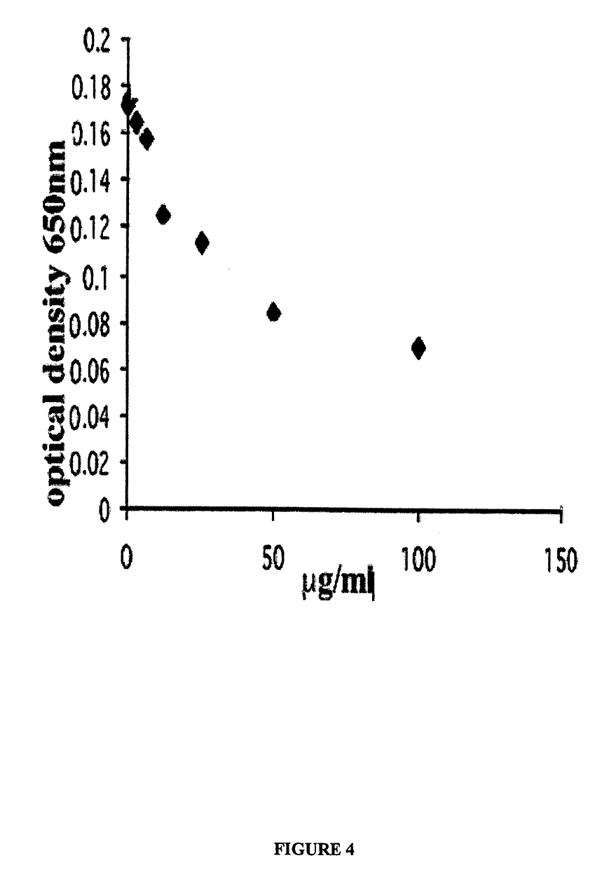

FIG. 4 corresponds to the inhibition of the growth of E. coli TG1 cells by the abovementioned compound 5,988,031. This figure represents the optical density measured at 650 nm (y-axis) with reference to the concentration of the compound 5,988,031 in μg/ml (x-axis).

The E. coli TG1 cells, diluted according to the protocol described hereafter in the experimental part, are incubated with increasing concentrations of the compound 5,988,031. The growth of the bacteria is measured at 650 nm after incubation for 12 hours at 37° C. under stirring in a microassay dish.

### EXPERIMENTAL PART

A subject of the invention is a new method which allows the screening of a zone of interaction of two proteins essential to the life of the cell in order to limit resistances by mutation of the target.

A subject of the invention is the screening of banks of synthetic chemical products or of natural products, even those contaminated by RNase or DNase activities.

Material and Methods

Detection of the sigma70-polymerase Bond

The sigma70 proteins and the RNA polymerase of E. coli are expressed and purified under standard conditions (Burgess et al., Biochemistry, 1975, October 21; 14(21): 4634-8; Burgess R., Methods Enzymol 1996; 273: 145-9). The sigma70 protein is stored at −80° C. in (20 mM tris HCl pH 8; 5 M guanidine chloride; 10 mM β-mercaptoethanol; 50% glycerol) at a concentration of 100 μM. The core RNA polymerase is stored at −80° C. in (20 mM tris HCl pH 8, 100 mM KCl, 10 mM β-mercaptoethanol, 50% glycerol) at a concentration of 1 μM. The antipolymerase monoclonal antibody was obtained and purified by standard techniques (Short Protocols in Molecular Biology 2^{nd} edition, John Wiley & Its) then chemically coupled to activated peroxidase (Boehringer Mannheim Biochemica ref. 1428861) according to the supplier's recommendations.

The test is based on the adsorption of sigma70 on an ELISA plate for 12 hours at 4° C. (6 pmoles of protein diluted in 100 μl of PBS per well, on Nunc-immuno plates, Maxisorp.). The protein dilutions and the buffers were optimized in order to reduce the non-specific bond of the RNA polymerase. After washing the plate with PBS 0.1% tween20, the plate is saturated with 200 μl of PBS 0.1% tween20, 1% BSA then incubated for 1 hour at ambient temperature with the RNA polymerase of E. coli (0.25 pmole of core RNA polymerase in 100 μl of PBS 0.1% tween 20, 1% BSA, 10 mM MgCl₂) in the presence or in the absence of an optional inhibitor. The plate is then washed, then incubated with an anti-RNA polymerase antibody coupled to peroxidase for 30 minutes at ambient temperature. The sigma70-RNA polymerase antibody complex is detected with a substrate of peroxidase, O-phenylenediamine, or another appropriate medium.

Alternatively, another protocol was used. This test is based on the adsorption of core RNA polymerase on an ELISA plate for 12 hours at 4° C. (0.5 pmoles of protein diluted in 100 μl of PBS per well on Nunc-immuno plates, Maxisorp). After washing the plate with PBS 0.1% tween20, the plate is saturated with 200 μl of PBS 0.1% tween20, 1% BSA, then incubated for 1 hour at ambient temperature with sigma70 comprising a C-terminal polyhistidine tag (1 pmole of sigma70 in 100 μl of PBS 0.1% tween20, 1% BSA, 10 mM MgCl₂) in the presence or in the absence of an optional inhibitor. The plate is then washed, then incubated with a polyhistidine tagged antibody coupled to peroxidase (Sigma ref. A7058 diluted 1/2000 (v/v)) for 30 minutes at ambient temperature. The sigma70-RNA polymerase antibody complex is detected with a substrate of peroxidase, O-phenylenediamine or another appropriate medium.

Detection of the NusA-polymerase Bond

The gene coding for the protein NusA and comprising a C-terminal polyhistidine tag was cloned in a pet21-type vector. After transfection in BL211amdaDE3 cells, the cells are cultured in LB medium at 37° C. under vigorous stirring. The production of protein is induced by the addition of 1 mM IPTG. After 3 hours' culture, the cells are recovered by centrifugation and lysed according to (Burgess et al., Biochemistry, 1975 October 21; 14(21): 4634-8). After centrifugation, the supernatant is passed over an Ni NTA agarose column (Quiagen) according to the supplier's recommendations. The protein NusA is stored at −80° C. in (20 mM tris HCl pH 8; 5 M guanidine chloride; 10 mM β-mercaptoethanol; 50% glycerol) at a concentration of 100 μM.

The test is based on the adsorption of NusA on an ELISA plate for 12 hours at 4° C. (6 pmoles of protein diluted in 100 μl of PBS per well on Nunc-immuno plates, Maxisorp.). The dilutions of protein and the buffers were optimized in order to reduce the non-specific bond of the RNA polymerase. After washing the plate with PBS 0.1% tween20, the plate is saturated with 2001 μl of PBS 0.1% tween20, 1% BSA, then incubated for 1 hour at ambient temperature with the RNA polymerase of E. coli (0.25 pmole of core RNA polymerase in 100 μl of PBS 0.1% tween20, 1% BSA, 10 mM MgCl₂) in the presence or in the absence of an optional inhibitor. The plate is then washed, then incubated with an anti-RNA polymerase antibody coupled to peroxidase for 30 minutes at ambient temperature. The sigma70-RNA polymerase-antibody complex is detected with a substrate of peroxidase, O-phenylenediamine, or another appropriate medium.

Preparation of the Reference Inhibitor and Antibody Used for the Detection

Mice are immunized with the RNA polymerase of E. coli. After 3 boosters with 100 μg, 50 μg and 10 μg of the RNA polymerase in the presence of Freund's complete adjuvant, the lymphocytes from the spleens of immunized mice are fused with the lymphoma cells. A group of 9 monoclonal antibodies is selected by ELISA using the RNA polymerase applied to plates. From these 9 antibodies the antibody 3E10 is obtained which is an inhibitor of the bond between the RNA polymerase and the protein σ70 or NusA, as well as the antibody 11D11, which recognizes the β′ sub-unit of the RNA polymerase, and which can serve to reveal the bond between the RNA polymerase and the protein σ70 or NusA.

The production of monoclonal antibodies from ascitic fluid is carried out according to the standard protocols (Short Protocols in Molecular Biology). The antibodies are purified by affinity chromatography on a protein A-sepharose column according to the reference “Short Protocols in Molecular Biology”.

Screening of Products

Primary Screening

A screening by competition between σ⁷, the core enzyme of the RNA polymerase of E. coli and the chemical compounds from a bank of 3200 molecules (Chembridge Inc.) was carried out. The protein σ⁷⁰, at a concentration of 1 μM in PBS buffer (150 mM NaCl; 2.5 mM K₂PO₄; 8.5 mM Na₂PO₄-pH 7.2), is adsorbed on a microassay plate overnight at 4° C. The plates are washed three times with 0.1% PBS-T (v/v) (150 mM NaCl; 2.5 mM K₂PO₄; 8.5 mM Na₂PO₄-pH 7.2; Tween 200.1% (v/v)) in order to eliminate anything not fixed on the plate. In order to avoid any non-specific reaction, the wells of the plate are then saturated with 200 μl of saturation solution (PBS-T 0.1% (v/v); 1% BSA (w/v)) for one hour at ambient temperature. After elimination of the saturation solution, the RNA polymerase and the optional competitors (at a concentration of 20 μg/ml) are incubated in these same wells for one hour at ambient temperature. The plates are then washed three times with 0.1% PBS-T (v/v). The bond between the core enzyme and the protein σ⁷⁰ is revealed by a monoclonal antibody to the β sub-unit of the RNA polymerase and coupled to peroxidase diluted to 1/2000^{th} (11D11) in the saturation buffer and incubated for 30 minutes at ambient temperature.

After three washings with 0.1% PBS-T (v/v), the peroxidase substrate is added (OPD, Biorad). The reaction takes place for 2 to 3 minutes in darkness then an absorbance measurement is carried out at 490 nm, after stopping the reaction with 50 μl of 4N H₂SO₄.

For the screening of inhibitors of the interaction between the core enzyme of the RNA polymerase and the protein NusA, the protocol is identical. However, the incubation stage with the potential inhibitors and the RNA polymerase is carried out in a 350 mM NaCl-2.5 mM K₂PO₄-8.5 mM Na₂PO₄; pH 7.2 mixture, in order to limit the non-specific bonds between the RNA polymerase and the protein NusA.

The compounds retained are those inhibiting more than 50% of the bond between the RNA polymerase and the protein NusA or σ⁷⁰, using for references:

a measurement without inhibitor, i.e. in the presence only of the RNA polymerase and of the protein intervening during the transcription, corresponding to the maximum bond,

a measurement in the presence of the RNA polymerase and of 100 pmole of σ⁷⁰ or of free NusA, corresponding to the minimum bond.

Thus, the following compounds were principally retained:

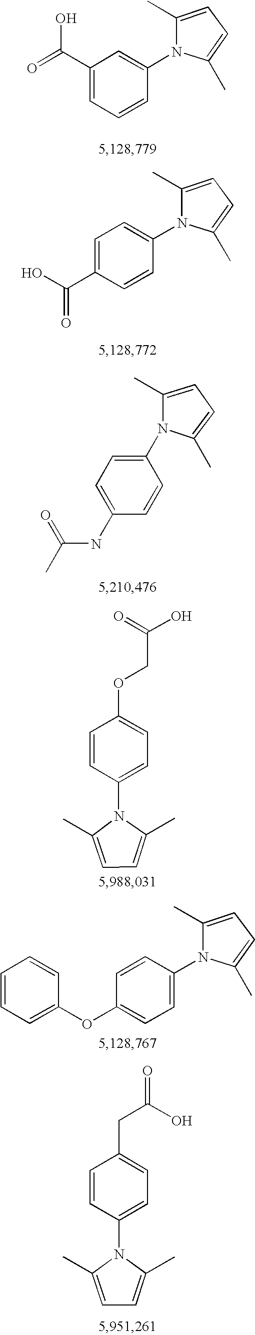

as well as the compounds of formulae:

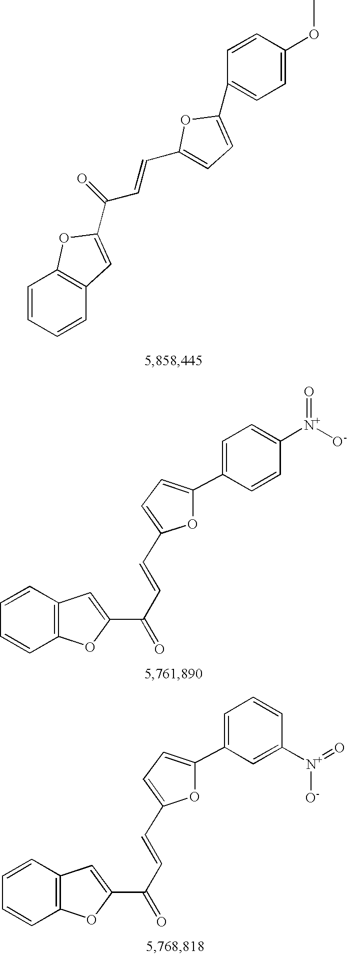

The compounds isolated at the end of the primary screening are subsequently tested at different concentrations (250; 165; 60; 30; 15; 7.5 and 3 μg/ml), according to the protocols described previously.

Anti-Bacterial Activity

a) Inhibition of Growth in Microassay Dish:

The various compounds isolated are tested for their TG1 E. coli bacteria growth-inhibiting properties. Cells in stationary phase are diluted to 1/10,000 in LB medium (tryptone 10 g; yeast extract 5 g; NaCl 5 g) and are transfected in a microassay dish (200 μl/well). The molecules are added to 10% DMSO, at a final concentration of 100 mg/ml. Under these conditions, the solvent does not affect the growth of the bacteria. After incubation overnight at 37° C. under stirring, the optical density of each well at 650 nm is measured.

b) Inhibition of Growth in Solid Medium:

A preculture of the strains E. coli K12, S. aureus and S. epidermis in Mueller Hinton Broth medium (Mueller and Hinton, 1941) is carried out at 37° C., until an optical density of 0.1 at 650 nm is obtained. 100 μl of the preculture is added to 10 ml of the medium: 0.1% agarose, 10 mM sodium phosphate pH 7.4; 0.3 mg/ml trypcase-soy; 100 mM NaCl.

The mixture is poured into a 10 mm Petri dish. Wells are made in the gelose and 5 μl of the solutions to be tested, containing the screened products, are placed in each well. The dishes are left at ambient temperature for 2 hours, then 10 ml of the medium (1% agarose; 10 mM sodium phosphate—pH 7.4-6% trypcase-soy) are poured into the Petri dish forming an overlay. After solidification, the dishes are incubated overnight at 37° C.

The antibacterial activity is evaluated by measuring the diameter of the bacterial growth inhibition of the zone at the centre of which the product was placed.

### CONCLUSION

The results of the bond tests clearly demonstrate that it is possible to detect compounds of low molecular weight which, to various degrees, inhibit the bond between the RNA polymerase and the protein NusA as well as the bond between the RNA polymerase and the protein σ⁷⁰ (see FIGS. 1, 2 and 3).

It is therefore noted that the two most active compounds in this bond test are the compounds 5,988,031 (FIGS. 1 and 3) and 5,858,445 (FIG. 2). In the case of the compound 5,988,031 (FIG. 1), a 50% inhibition of the bond between σ⁷⁰ and the RNA polymerase is observed for a σ⁷⁰ concentration of approximately 5 μg/ml. It is also noted that three analogues of said compound, namely the compounds 5,951,261, 5,128,773 and 5,128,772, are also effective at concentrations 5 to 10 times higher. Thus, the position and the length of the chain which carry the carboxylic acid bonded to the benzene ring of the product 5,988,031 strongly influence the apparent affinity of these molecules.

The two families of molecules tested, namely those of the compound 5,988,031 and 5,858,445, displace both the core enzyme—σ⁷⁰ bond and the core enzyme—NusA bond. The bond of these two proteins with the RNA polymerase being mutually exclusive (which signifies that the two proteins are in competition for the same binding site), it is not surprising that the two inhibitors tested displace the two proteins. However, the possibility of isolating specific inhibitors of the bond between the RNA polymerase and one or other of these proteins cannot be excluded.

The compound 5,988,031 inhibits the bond between σ⁷⁰ and the RNA polymerase, the bond between NusA and the RNA polymerase, but not the bond between an antibody, for example 11D11, and the RNA polymerase or the assembly of the α, β and β′ subunits. These results therefore make it possible to demonstrate the specificity of this compound.

Moreover, the antibiotic activities of these different molecules have been evaluated on target bacteria.

Thus, the compound 5,988,031 inhibits the growth of relatively sensitive cells such as E. coli TG1, which demonstrates the direct link between the bond test and the biological activity (see FIG. 4). However, it has been noted that this molecule is not active on other strains tested, such as S. aureus, E. coli K12, M. Luteus etc.

The table, represented above, correspond to the results obtained during the bacterial activity tests and indicates for each compound tested the diameter of inhibition measured.

| | K12 | S epidermidis | S aureus |
|---|---|---|---|
| 5,858,445 | negative | 4.2 mm | 4.2 mm |
| 500 μg/ml | | | |
| 5,761,890 | negative | negative | negative |
| 500 μg/ml | | | |
| 5,768,818 | negative | negative | negative |
| 500 μg/ml | | | |
| vancomycin | negative | 5 mm | 5 mm |
| 50 μg/ml | | | |

Compound 5,858,445 also strongly inhibits the growth of S. aureus and S. Epidermidis bacteria cells in the solid medium test (see anti-bacterial activity). An inhibition diameter (cf. anti-bacterial activity) of 4.2 mm is observed at a concentration of 500 μg/ml, whereas, under these conditions, an inhibition diameter of 5 mm is observed with vancomycin at 50 μg/ml.

It is moreover noted that the analogues of this compound 5,858,445 have no effect with respect to the anti-bacterial activity. It therefore appears that the replacement of the nitro group by a methoxy group is important for the anti-bacterial properties of the compounds but not for the properties of inhibition of the bond between the RNA polymerase and a protein intervening during the transcription.

These results make it possible to demonstrate the link between the bond test activity and the biological activity. Moreover, results obtained from the bond test with E. coli proteins make it possible to target molecules active on other pathogenic bacteria which often have an RNA polymerase having strong homologies with that of E. coli, i.e. an identity percentage of approximately 90% at the level of the σ regions involved in the bond with the RNA polymerase.

### REFERENCES

Burgess et al. (1969) Nature 221, 43-44,

Burgess et al. (1975) Biochemistry, Oct 21; 14(21): 4634-8,

Burgess (1996) Methods Enzymol 273: 145-9,

Courvalin (1996) Antimicrob Chemother 37, 855-69,

Kolesky et al. (1999) J Mol Biol 291, 267-81,

Lesley et al. (1989) Biochemistry 28, 7728-7734,

Mueller, J. H. and Hinton, J. (1941) A protein-free medium for primary isolation of gonococcus and meningococcus, Proc. Soc. Exp. Biol. Med. 48, 330-333,

Reznikoff et al. (1985) Annu. Rev. Genet. 19, 355-387,

Wu et al. (1997) Analytical Biochemistry 245, 226-230,

Yang et al. (1995) J Biol Chem 270, 23930-3,

Short Protocols in Molecular Biology, 2^{nd} Edition, John Wiley & Its.

1. Process for the detection of a compound modulating the complexation between RNA polymerase and a protein intervening during the transcription, in which:
a mixture comprising the RNA polymerase, a protein intervening during the transcription and the compound subjected to the detection process is incubated, the incubation stage being carried out under conditions allowing:
the formation of a complex between the RNA polymerase and said protein and,
the formation of a bond, on the one hand between said compound and on the other hand the RNA polymerase, and/or the protein intervening during the transcription,
by means of a complexation test, any significant variation in the quantity of complex formed between the RNA polymerase and said protein, with respect to a control value corresponding to the quantity of complex formed between the RNA polymerase and said protein in the absence of any modulator is detected, and
when there is a significant variation as defined above, it is deduced from this, that a bond has been formed between on the one hand said compound and on the other hand the RNA polymerase, and/or the protein intervening during the transcription, which is translated by a modulation of the complexation between the RNA polymerase and the protein intervening during the transcription.

2. Detection process according to claim 1, in which the modulating compound is a compound activating the complexation between the RNA polymerase and a protein intervening during the transcription, and in which:
a mixture comprising the RNA polymerase, a protein intervening during the transcription and the compound subjected to the detection process is incubated, the incubation stage being carried out under conditions allowing:
the formation of a complex between the RNA polymerase and said protein and,
optionally the formation of a bond on the one hand between said compound and on the other hand the RNA polymerase, and/or the protein intervening during the transcription,
by means of a complexation test, any significant variation in the quantity of complex formed between the RNA polymerase and said protein, with respect to a control value corresponding to the quantity of complex formed between the RNA polymerase and said protein in the absence of any activator is detected, and
when there is a significant variation as defined above, it is deduced from this, that a bond has been formed between on the one hand said compound and on the other hand the RNA polymerase, and/or the protein intervening during the transcription, which is translated by an activation of the complexation between the RNA polymerase and the protein intervening during the transcription.

3. Detection process according to claim 1, in which the modulating compound is a compound inhibiting the complexation between the RNA polymerase and a protein intervening during the transcription, and in which:
a mixture comprising the RNA polymerase, a protein intervening during the transcription and the compound subjected to the detection process is incubated, the incubation stage being carried out under conditions allowing:
the formation of a complex between the RNA polymerase and said protein and,
optionally the formation of a bond on the one hand between said compound and on the other hand the RNA polymerase, and/or the protein intervening during the transcription,
by means of a complexation test, any significant variation in the quantity of complex formed between the RNA polymerase and said protein, with respect to a first control value and/or to a second control value is detected, one of these control values corresponding to the quantity of complex formed between the RNA polymerase and said protein in the absence of any inhibitor and the other of these control values corresponding to the quantity of complex formed between the RNA polymerase and said protein in the presence of a reference inhibitor, and
when there is a significant variation as defined above, it is deduced from this, that a bond has been formed between on the one hand said compound and on the other hand the RNA polymerase, and/or the protein intervening during the transcription, which is translated by an inhibition of the complexation between the RNA polymerase and the protein intervening during the transcription.

4. Detection process according to claim 3, comprising the use of two control values, one corresponding to the incubation of the RNA polymerase alone with the protein intervening during the transcription in the absence of any inhibitor (which corresponds to an absence of inhibition) and the other corresponding to an incubation of the RNA polymerase with the protein intervening during the transcription and with a reference inhibitor (which corresponds to a reference inhibition).

5. Detection process according to claim 3, in solid phase, comprising the use of a first control value and/or of a second control value and/or of a third control value, one corresponding to the quantity of complex formed between the RNA polymerase and said protein in the absence of any inhibitor, the other corresponding to the quantity of complex formed between the RNA polymerase and said protein in the presence of a reference inhibitor, and the other corresponding:
either to the absence of complex formed between the RNA polymerase and the protein intervening during the transcription, resulting from the introduction of the protein intervening during the transcription fixed on a support, of the RNA polymerase and of an excess of protein intervening during the transcription, the excess of protein intervening during the transcription preferably being previously incubated with the RNA polymerase, the absence of said complex corresponding to a total inhibition of the complexation between the RNA polymerase and said protein,
or to the absence of complex formed between the RNA polymerase and the protein intervening during the transcription, resulting from the presence of RNA polymerase alone and from the absence of protein intervening during the transcription.

6. Detection process according to claim 3, in liquid phase, comprising the use of a first control value and/or of a second control value and/or of a third control value, one corresponding to the quantity of complex formed between the RNA polymerase and said protein in the absence of any inhibitor, the other corresponding to the quantity of complex formed between the RNA polymerase and said protein in the presence of a reference inhibitor, and the other corresponding to the absence of complex formed between the RNA polymerase and the protein intervening during the transcription, resulting from the introduction of the RNA polymerase, of an excess of non-marked protein intervening during the transcription and of said marked protein, the absence of said complex corresponding to a total inhibition of the complexation between the RNA polymerase and said protein.

7. Detection process according to one of claims 1 to 6, in which the mixture comprising the RNA polymerase, a protein intervening during the transcription and a compound subjected to the detection process is prepared:
either by simultaneously adding the RNA polymerase, the protein intervening during the transcription and the compound subjected to the detection process,
or by successively adding: the RNA polymerase, the compound subjected to the detection process and the protein intervening during the transcription, or successively: the protein intervening during the transcription, the compound subjected to the detection process and the RNA polymerase,
or by adding said compound previously incubated with the RNA polymerase or said protein, and either said protein or the RNA polymerase respectively,
or by adding said compound and the RNA polymerase previously incubated with said protein.

8. Detection process according to one of claims 1 to 7, in which, before, during or after the incubation stage, either the RNA polymerase or the protein intervening during the transcription is applied to a solid support.

9. Detection process according to one of claims 1 to 8, in which, during the incubation stage of the RNA polymerase with a protein intervening at the time of the transcription and with a compound subjected to the detection process, a bond is formed:
either between said compound and between the RNA polymerase,
or between said compound and between said protein,
or between said compound, between said protein and between the RNA polymerase.

10. Detection process according to one of claims 1 to 9, in which, during the stage of detection of any significant variation in the quantity of complex formed between the RNA polymerase and the protein intervening during the transcription, an anti-RNA polymerase antibody is used.

11. Detection process according to one of claims 1 to 10, in which the protein intervening during the transcription has a molecular weight greater than approximately 15 kDa or is a fusion protein between a protein with a molecular weight of less than 15 kDa and another protein such as GST (glutathione S transferase).

12. Detection process according to one of claims 1 to 11, in which the RNA polymerase concentrations are comprised between approximately 1 fmole and approximately 100 pmole/test, in particular between approximately 1 fmole and approximately 10 pmole/test, those of the protein intervening during the transcription between approximately 10 fmole and approximately 500 pmole/test and those of the compound subjected to the detection process between approximately 1 μM and approximately 1 μM, in particular between approximately 1 nM and approximately 1 μM.

13. Detection process according to one of claims 1 to 12, in which the RNA polymerase used originates from prokaryotic cells, in particular from E. coli.

14. Detection process according to one of claims 1 to 13, in which the protein intervening during the transcription intervenes either during the transcription initiation stage, or during the elongation stage, or during the transcription termination stage.

15. Detection process according to one of claims 1 to 14, in which the protein intervening during the transcription is:
either the protein σ⁷⁰ or an equivalent protein,
either the protein NusA or an equivalent protein,
either fragments of one of these two proteins,
or one of these two proteins in the form of a fusion protein,
or fragments of one of these two proteins in the form of a fusion protein.

16. Kit for the detection of a modulating compound, in particular a compound inhibiting the complexation between the RNA polymerase and a protein intervening during the transcription comprising:
one or more proteins intervening during the transcription; said protein can be in the form of a fusion protein and is in particular:
either the protein σ⁷⁰ or an equivalent protein,
or the protein NusA or an equivalent protein,
or fragments of one of these two proteins,
the RNA polymerase,
media or buffers necessary for dilution,
optionally washing means,
media or buffers allowing the formation of a complex between the RNA polymerase and the protein intervening during the transcription and the formation of a bond between the RNA polymerase and the modulating compound,
means for the detection of the variation in the quantity of complex formed between the RNA polymerase and between the protein intervening during the transcription.
